(19) United States
(12) Patent Application Publication
(10) Pub. No.: US 2010/0122359 A1
(43) Pub. Date: May 13, 2010

(54) Sex Selected Equine Intracytoplasmic Sperm Injection Embryo Production System

(75) Inventors: Tae Kwang Suh, Fort Collins, CO (US); Edward L. Squires, Nicholasville, KY (US); Elaine Carnevale, Loveland, CO (US); James K. Graham, Fort Collins, CO (US); Juan F. Moreno, College Station, TX (US)

Correspondence Address: CR MILES P.C., 405 Mason Court, Suite 119, Fort Collins, CO, 80524, US

(73) Assignee: XY, Inc. (CSURF)

(21) Appl. No.: 12/452,695

(22) PCT Filed: Jul. 18, 2008
(86) PCT No.: PCT/US08/08772

§ 371 Date: Jan. 15, 2010

Related U.S. Application Data
(60) Provisional application No. 60/961,335, filed on Jul. 19, 2007.

(51) Int. Cl. A01K 67/00; A01N 1/02; A61B 17/43

(52) U.S. Cl. 800/8; 600/35; 435/2

(57) ABSTRACT

Intracytoplasmic sperm injection utilizing sex-selected equine spermatozoa to obtain viable sex selected embryos transferable to a recipient female equine mammal to obtain sex selected foals.

This United States National Stage patent application is a continuation-in-part of International Patent Cooperation Treaty Application No. PCT/US2008/008772, filed Jul. 18, 2008, and claims the benefit of U.S. Provisional Patent Application No. 60/961,335, filed Jul. 19, 2007, each hereby incorporated by reference herein.

### I. Technical Field

Intracytoplasmic sperm injection utilizing sex-selected equine spermatozoa to obtain viable sex selected embryos transferable to a recipient female equine mammal to obtain sex selected foals.

### II. BACKGROUND

Artificial insemination (AI) and embryo transfer (ET) are conventional assisted reproductive technologies (CART) utilized in breeding equine mammals. However, conventional AI and ET can only be utilized for mares or stallions with normal reproductive function. Unfortunately, approximately ten percent of the equine population is estimated to have reproduction impaired to the extent which precludes the use AI and ET.

As to this part of the equine population, oocyte transfer (OT) and intracytoplasmic sperm injection (ICSI) may be an alternative to CART. However, prior to the present invention, it is believed that ICSI has not been successful when utilized with oocytes matured in vivo and has not been utilized with stallion spermatozoa which has been sex-selected utilizing flow cytometry (or other sex selection means or methods) which sorts equine spermatozoa (also referred to herein as equine sperm cells) entrained in droplets based on the amount of DNA contained within each equine sperm cell into an X-chromosome bearing and a Y-chromosome bearing populations, as further described below. It is believed that no method of equine ICSI prior to the instant invention has produced any live foal.

A significant problem with the use of equine semen obtained from various members of the Equidae family (including without limitation horses, donkeys, zebras, burros, asses, tarpan, quagga, or the like) in conjunction with CART and in particular equine spermatozoa which have been sex-selected in conjunction with CART, or equine spermatozoa which have been frozen prior to the application of CART (whether or not sex-selected) and specifically with respect to the application of ICSI with such equine spermatozoa can be that the equine spermatozoa are no longer viable and cannot be utilized to fertilize oocytes whether in vivo or in vitro and specifically have not been successfully utilized in ICSI methods to fertilize oocytes, or the resulting embryos have not been viable and cannot be utilized for ET. The frail nature of equine spermatozoa is well known and as to any method which utilizes equine spermatozoa for the production of viable embryos it cannot be predicted that a particular method will be successful or whether a method will produce comparable results to controls in advance of the actual reduction to practice.

The instant invention provides methods of utilizing equine spermatozoa and sex-selected equine spermatozoa with ICSI for the fertilization of oocytes and production of viable embryos which can be transferred to recipient animals for the production of live foals to addressee the significant problems with CART for the breeding of equids in general and specifically for that part of the equine population having reproduction impaired to the extent which precludes the use AI or ET or both AI and ET.

### III. DISCLOSURE OF INVENTION

Accordingly, a broad object of the invention can be to provide methods of equine oocyte collection, equine semen preparation, intracytoplasmic injection (ICIS), embryo culture, and embryo transfer which can be used in combination to produce live foals, or can be used independently of one another to provide viable oocytes, viable equine semen, viable fertilized oocytes, viable embryos, viable implanted embryos, and live foals.

Another broad object of the invention can be to provide methods of using sex-selected equine spermatozoa in conjunction with ICSI to produce viable sex-selected fertilized equine oocytes, viable sex selected equine embryos, and viable sex-selected live foals.

Naturally, further objects of the invention are disclosed throughout other areas of the specification, drawings, photographs, and claims.

### IV. BRIEF DESCRIPTION OF THE DRAWINGS

FIG. 1 provides an illustration of a flow cytometer utilized to determine the sex of a plurality of equine sperm cells and sort the plurality of equine sperm cells based upon the determined sex into an X-chromosome bearing population and a Y-chromosome bearing population of sex-selected equine sperm cells and further provides a block diagram of the steps in producing a live equine ICSI sex-selected foal.

FIG. 2 shows a particular embodiment of a viewable data representation generated by a particular embodiment of the flow cytometer shown in FIG. 1 which shows the separation of the plurality of equine sperm cells based upon the determined sex into an X-chromosome bearing population and a Y-chromosome bearing population of sex-selected equine sperm cells.

FIG. 3 shows a particular embodiment of a viewable data representation generated by a particular embodiment of the flow cytometer shown in FIG. 1 which shows the separation of the plurality of equine sperm cells based upon the determined sex into an X-chromosome bearing population and a Y-chromosome bearing population of sex-selected equine sperm cells.

### V. MODE(S) FOR CARRYING OUT THE INVENTION

Intracytoplasmic sperm injection utilizing sex-selected equine spermatozoa to obtain viable sex selected embryos transferable to a recipient female equine mammal to obtain sex selected foals.

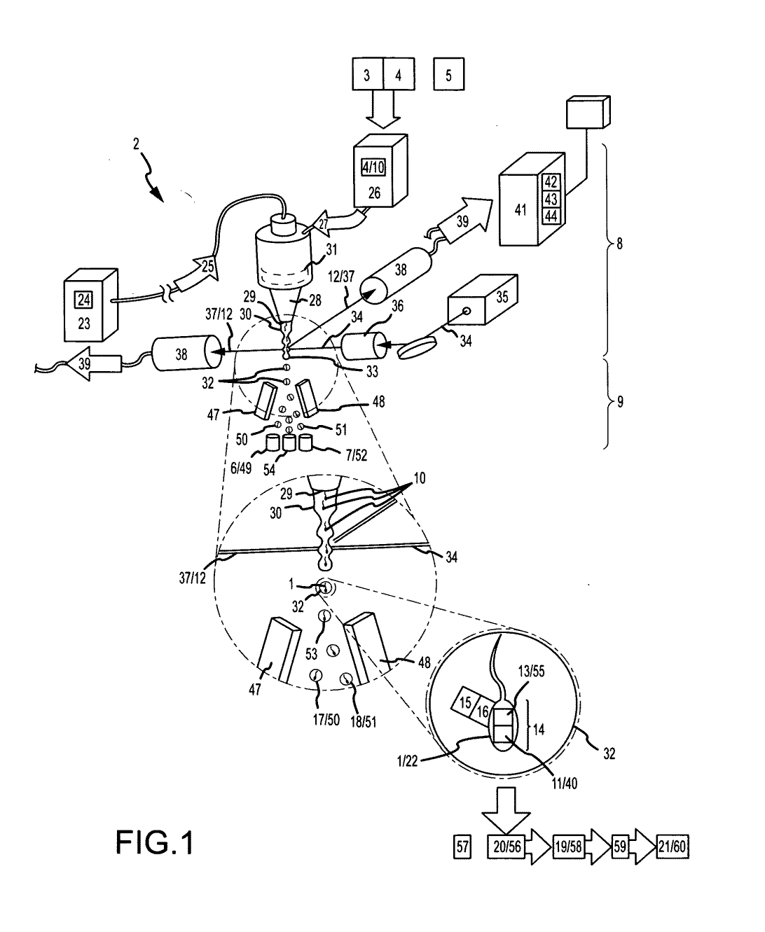

Now referring primarily to FIG. 1 a non-limiting example of a device for the production of sex-selected sperm cells (1) in the form of a flow cytometer (2) is shown. For the purposes of this invention the term “sperm cells” means spermatozoa obtained from a male mammal (3) and without limitation includes non-human male mammals such as a bovid, an ovis, an equid, a pig, a cervid, a canid, a felid, a rodent, a whale, a rabbit, an elephant, a rhinoceros, a primate, or the like, and specifically includes equine sperm cells (4) obtained from an equine male mammal (5) of the Equidae family (including for example without limitation horses, donkeys, zebras, burros, asses, tarpan, quagga, or the like). Also, for the purposes of this invention “sex-selected” means a population separated into an X-chromosome bearing population (6) and a Y-chromosome bearing population (7) regardless of the differentiation means (8) or separation means (9) utilized and specifically with regard to sex-selected sperm cells (1) means the product of separating sperm cells based on differentiating or determining sex (the presence or absence of an X chromosome or a Y chromosome) of each of a plurality of sperm cells (10) regardless as to whether differentiation is based upon amount of deoxyribonucleic acid (DNA) (11) or a part of an amount of DNA, amount of fluorescence (12) of a DNA selective material (13) substantially quantitatively bound to an amount of DNA (11) or to a part of the amount of DNA (11), greater or lesser volume of the sperm head (14), optical trapping, optical force trap, optical tweezers, greater or lesser density, motility, a protein selective material (15) such as an antibody bound to a protein (16) or part of a protein, or the like, and specifically includes the product of differentiation means and separation means of an isolated sperm cell population in which substantially all of the plurality of sperm cells (10) are X-chromosome bearing sperm cells (17) or Y-chromosome bearing sperm cells (18) but also includes the product of differentiation means and separation means of an isolated X-chromosome bearing sperm cell population (6) or Y-chromosome bearing sperm cell population (7) which has a substantially greater percentage of either X-chromosome bearing sperm cells (17) or Y-chromosome bearing sperm cells (18) as compared to the original plurality of sperm cells (10) prior to differentiation such as 60%, 70%, 80%, 90%, 95%, 98% of either X-chromosome bearing sperm cells (17) or Y-chromosome bearing sperm cells (18) in sufficiently viable condition to fertilize an oocyte (20) (whether live or dead whole sperm, part of a live sperm, tail-less sperm, immobilized sperm, sperm heads, or the like) and specifically with respect sex-selected embryos (19) means an oocyte (20) or a population of oocytes fertilized with a sex-selected sperm cell (1) or an embryo that results from fertilization of an oocyte (20) with a sex-selected sperm cell (1). Also for the purposes of this invention the term “selected sex” means selection of a sex for an sex-selected embryo (19) or a sex-selected offspring animal (21) by use of sex-selected sperm cells (1) to fertilize an oocyte(s) (20) whether matured in vivo or in vitro whether by artificial insemination, in vitro fertilization, or ICSI, or otherwise. Also, for the purposes of the present invention, ranges may be expressed herein as from “about” one particular value to “about” another particular value. When such a range is expressed, another embodiment includes from the one particular value to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Now referring primarily to FIG. 1, a non-limiting embodiment of a differentiation means (8) and a separation means (9) can include a flow cytometer (2) capable of producing sex-selected sperm cells (1) and specifically sex selected equine sperm cells (22). Embodiments of the flow cytometer (2) can provide a fluid source (23) which supplies a sheath fluid (24) to establish a sheath fluid stream (25). A sperm cell source (26) can entrain a plurality of sperm cells (10) (and specifically can entrain a plurality equine sperm cells (4)) in a sample fluid stream (27). The sample fluid stream (27) entraining the plurality of sperm cells (10) joins the sheath fluid stream (25) in the nozzle (28) of the flow cytometer (2) as coaxial laminar flow with the sample fluid stream (27) surrounded by the sheath fluid stream (25). The coaxial laminar flow exits the nozzle orifice (29) as a fluid stream (30) entraining the plurality of sperm cells (10).

The nozzle (28) can be made responsive to an oscillator (31) (see FIG. 1 broken lines). Oscillation of the nozzle (28) can perturb the fluid stream (30) to establish a steady state oscillation of the fluid stream (30). One non-limiting example of an oscillator (31) capable of perturbing the fluid stream (30) directly or indirectly by oscillation of the nozzle (28) is a piezoelectric crystal. The oscillator (31) may have an adjustable oscillation frequency that can be adjusted to perturb the fluid stream (30) at different frequencies. Steady state oscillation of the fluid stream (30) can be established in a condition such that droplets (32) are formed and break away from a contiguous part of the fluid stream (30). When the fluid stream (30) is established in this steady state fashion, a stable droplet break-off point (33) can be established.

The fluid stream (30) in steady state oscillation can be interrogated with one or more light beams (34) such as one or more a laser beams emitted from a light emission source (35). The one or more light beams (34) can pass through a beam shaping optics (36) to configure the shape of the light beams (34) and focus the light beams (34) on the fluid stream (30). An amount of light (37) emitted, fluoresced (12) or reflected from one of the plurality of sperm cells (10) in the interrogated fluid stream (30) can be received by a photoreceiver (38). The photoreceiver (38) converts the received amount of light (37) into a signal (39) (whether analog, analog converted to digital, or digital) which varies whether in frequency, amplitude, or both frequency and amplitude) based upon differences in at least one sperm cell characteristic (40) among the plurality of sperm cells (10). The term “at least one sperm cell characteristic” for the purposes of this invention means at least one part, component, or differentially modified part or component common to at least a portion of the plurality of sperm cells (10) entrained in the fluid stream (30) which varies in kind or amount between the plurality of sperm cells (10) which allows differentiation of the plurality of sperm cells (10) based on the sex (whether it is an X-chromosome bearing sperm cell (17) or Y-chromosome bearing sperm cell (18)).

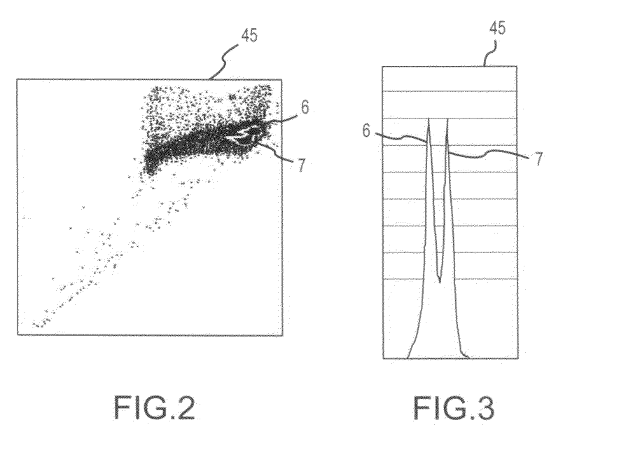

Now referring primarily to FIGS. 1 and 2, the flow cytometer (2) can further include a computer (41) which executes the functions of a sperm cell analysis application (42) which in part provides a signal analyzer (43) which intermittently or continuously converts the signal (39) produced by interrogation of the fluid stream (30) into a data representation (44) of occurrence or detection of at least one sperm cell characteristic (40) in the plurality of sperm cells (10) interrogated. The data representation (44) can be continuously or intermittently displayed as a viewable data representation (45) (see for example FIGS. 2A and 2B) on a monitor (46) or updated upon elapse of a short interval of time such as 100 milliseconds.

Certain embodiments of the signal analyzer (43) can further function to establish parameters and timed events by which the plurality of sperm cells (10) can be separated, parsed or divided based upon the presence, absence, or amount of the at least one sperm cell characteristic (40). A flow cytometer (2) such as a MOFLO® SX can used to separate or sort the plurality of sperm cells (10) into, discreet sub-populations based upon at least one sperm cell characteristic (40). Subsequent to exiting the nozzle (28), the fluid stream (30) breaks into droplets (32) each of which can contain a corresponding one each of the plurality of sperm cells (10). Based on the above-described analysis of each of the plurality of sperm cells (10) in the fluid stream (30), the droplets (32) can be differentiated based on the at least one sperm cell characteristic (40) and separated by applying a charge (whether positive or negative) to each one of the droplets (32) analyzed and then deflecting the trajectory of each of the droplets (32) by passing the droplets (32) through a pair of charged plates (47)(48). The trajectory of the positively charged droplets (50) can be altered for delivery to a first container (49) and the trajectory of the negatively charged droplets (51) can be altered for delivery to a second container (52) (each the first container and the second container a discrete container). Uncharged droplets (53) are not deflected and can be delivered to a third container (54) or to a waste stream.

As one non-limiting example, the plurality of sperm cells (10) can be a plurality of equine sperm cells (4) and the at least one particle characteristic (40) can be the amount of deoxyribonucleic acid (“DNA”) (11) contained in each of the plurality of equine sperm cells (4). The amount of DNA (11) can vary based upon whether the particular one of the plurality of equine sperm cells (4) is an X chromosome bearing sperm cell (17) or a Y chromosome bearing sperm cell (18). The X chromosome contains a greater amount of DNA (11) than the corresponding Y chromosome in the plurality of equine sperm cells (4) obtained from the equine male mammal (5). The amount of DNA (11) in each of the plurality of equine sperm cells (4) can be stained with a DNA selective stain (55) for a period of time to substantially uniformly stain the amount of DNA (11) while limiting the period of time of the staining procedure to maintain viability of a portion of the plurality of equine sperm cells (4). A non-limiting example of DNA stains (55) which are membrane permeant stains include without limitation: SYTO 40 blue-fluorescent nucleic acid stain, SYTO 41 blue, SYTO 42 blue, SYTO 43 blue, SYTO 44 blue, SYTO 45 blue, a green-fluorescent SYTO dye, SYTO 9 green, SYTO 10 green, SYTO BC green, SYTO 13 green, SYTO 16 green, SYTO 24 green, SYTO 21 green, SYTO 27 green, SYTO 26 green, SYTO 23 green, SYTO 12 green, SYTO 11 green, SYTO 20 green, SYTO 22 green, SYTO 15 green, SYTO 14 green, SYTO 25 green, an orange-fluorescent SYTO dye, SYTO 86 orange, SYTO 81 orange, SYTO 80 orange, SYTO 82 orange, SYTO 83 orange, SYTO 84 orange, SYTO 85 orange, a red-fluorescent SYTO dye, SYTO 64 red, SYTO 61 red, SYTO 17 red, SYTO 59 red, SYTO 62 red, SYTO 60 red, SYTO 63 red, a Hoechst dye, Hoechst 33342, Hoechst 34580, Hoechst 33258, DAPI, LDS 751 and dihydroethidium.

Additionally, certain DNA stains (55) are membrane impermeant including without limitation: SYTOX blue, SYTOX green, SYTOX orange, a cyanine dimer, POPO-1, BOBO-1, YOYO-1, TOTO-1, JOJO-1, POPO-3, LOLO-1, BOBO-3, YOYO-3, TOTO-3, a cyanine monomer, PO-PRO-1, BO-PRO-1, YO-PRO-1, TO-PRO-1, JO-PRO-1, PO-PRO-3, LO-PRO-1, BO-PRO-3, YO-PRO-3, TO-PRO-3, TO-PRO-5, acridine homodimer, 7-amino actinomycin D, ethidium bromide, ethidium homodimer-1, ethidium homodimer-2, ethidium nonazide, nuclear yellow and propidium iodide.

Electroporation can be utilized to temporarily destabilize the membrane of the plurality of sperm cells (10) by exposure to short, high intensity electric field pulses which can make the cell membrane highly permeable to DNA selective materials (13) present in the surrounding media such as certain DNA stains (55).

Now referring primarily to FIG. 1, a plurality of equine sperm cells (4) for use in equine sex-selected ICSI can be obtained by collection of the ejaculate of a equine male mammal (5). The ejaculate can be diluted with Kenney's extender supplemented with a modified high-potassium Tyrode's medium (KMT) and centrifuged at 600 g for 10 min. The supernatant can be removed, sperm concentration in the remaining pellet can be determined by hemacytometer and the plurality of equine sperm cells (4) can then be resuspended to a final concentration of about 400×10⁶ equine sperm cells/mL in KMT. The plurality of equine sperm cells (4) can be substantially uniformly stained for flow cytometer (2) sorting at about 34° C. for about 30 minutes by mixing about 10.54 μL Hoechst 33342, about 1.489 mL KMT, and about 500 μL of the suspension of the plurality of equine sperm cells (4). KMT with food dye (FD&C #40) can be warmed and added to the stained plurality of equine sperm cells (4) at a volume of about 0.75 μl/ml of 5% red food dye. Stained equine sperm cells (4) can be filtered using yellow Partec filters and incubated at a temperature in a range of about 20-22° C. until use.

The stained equine sperm cells (10) can be sorted as described above and in response to interrogation with the light beam(s) (34) such as a laser beam the DNA selective stain (55) bound to the amount of DNA (11) contained each of the plurality of equine sperm cells (4) can emit an amount of light (37). X chromosome bearing sperm cells (17) typically emit a greater amount of light (37) than Y chromosome bearing sperm cells (18) because each X chromosome bearing sperm cell (17) contains a greater amount of stained DNA (11) than a Y chromosome bearing sperm cell (18). The photoreceiver (38) can convert the amount of light (37) (or fluorescence) into a signal (39) which correspondingly varies based upon the difference in the amount of light (37) emitted by X chromosome bearing equine sperm cells (17) and Y chromosome bearing equine sperm cells (18) when passed through the light beam (34). With respect to the separation of a plurality of equine sperm cells (4), the separated sub-populations can include X chromosome bearing equine sperm cells (17) isolated in the first container (49) and Y chromosome bearing equine sperm cells (18) isolated in the second container (52).

Two ml of egg-yolk containing semen extender (FR4) can be warmed to a temperature of between about 20° C. to about 22° C. and transferred into a 50 mL tube as a the first container (49) in which to collect sorted X-chromosome bearing equine sperm cells (17). A similar second container (52) can be provided in which to collect sorted Y-chromosome bearing equine sperm cells (18). The flow cytometer (2) sorting gates can be set to allow collection of X-chromosome bearing equine sperm cells (17) and Y-chromosome bearing equine sperm cells (18) at about 90% purity (or other lesser or greater desired purity) with a sorting volume of up to 15-mL per collection tube. The sex-selected equine sperm cells (22) can be swirled about every 20 minutes in the first collection container (49) or after sort of about each 500,000 sperm.

The sex-selected equine sperm cells (22) in the first container (49) (or the second container (52) depending on the sex of the sex-selected equine sperm cells (22) collected can be centrifuged at 850×g for 20 minutes, the supernatant aspirated leaving a pellet of about 100 μL of equine sex-selected sperm cells (22), and 100 μL of glycerol containing semen extender (FR5) was added to each pellet of equine sex-selected sperm cells (22). The first container (49) containing a pellet of sex-selected sperm cells (22) was put in a beaker containing 300 ml of room temperature water, and cooled to 5° C. for 90 minutes. The sperm concentration can be calculated by using hemacytometers, and the final sex-selected equine sperm cell (22) concentration can be adjusted to about 87×10⁶ sperm/mL by adding FR5. Sorted sex-selected equine sperm cells (22) can be loaded into each 0.25 ml straw and the open end of each straw sealed by use of metal balls inserted in each end of the straw (as one example of sealing the straw). Straws can be placed on a pre-cooled freezing rack, and the rack can be placed in nitrogen vapor at approximately −100° C. After allowing 5 minutes for freezing, straws can be plunged into liquid nitrogen for long-term storage. See also, U.S. Pat. No. 6,149,867, which is hereby incorporated by reference herein.

A plurality of equine sperm cells (4) utilized as comparative controls to sex-selected equine sperm cells (22) can be obtained as male equine mammal (5) ejaculate diluted to a concentration of about 50×10⁶ sperm/mL in a skim milk, glucose diluent (EZ-Mixin, Animal Reproduction Systems, Chino, Calif.), and centrifuged at about 600 g for about 10 min. The supernatant can then removed, the equine sperm cell (4) concentration in the remaining pellet can be determined by hemacytometer, and the equine sperm cells (4) can be resuspended to a final concentration of 400×10⁶ sperm/ml in Lactose-EDTA freezing extender containing 5% glycerol (EZ-Freezin, Animal Reproduction Systems, Chino, Calif.). The equine sperm cells (4) can then be packaged into 0.5 cc straws and frozen in a programmable freezer (Kryo 10 Series III, Planer, Middlesex, UK) at a rate of about −10° C./min from 20 to −15° C. and then about −15° C./min from −15 to −120° C. At −120° C., straws can be plunged into liquid nitrogen and stored for use as comparative controls for equine ICSI.

The sex-selected equine sperm cells (22) (along with the comparative controls) frozen as above described can be can be prepared for equine ICSI by washing the frozen sex-selected equine sperm cells (22). The frozen sex-selected equine sperm cells (22) can be washed by transferring about a 25 μL part of a frozen straw containing sex-selected equine sperm cells (22) into the bottom of 15 ml centrifugation tube containing 2 ml of FCDM, and washed by centrifugation at about 300 g for about 5 min. The supernatant can be removed, and the pellet placed in an incubator until use.

Again referring primarily to FIG. 1, as an alternative to or in addition to the step of washing the sex-selected equine sperm cells (22) as above described to obtain sex-selected equine sperm cells (22) for equine ICSI (also to obtain comparative controls), a swim-up step (also referred to as swimming-up) of sex-selected equine sperm cells (22) and equine sperm cells (4) can be performed by placing a 25 μl part of a frozen straw containing sex-selected equine sperm cells (22) or equine sperm cells (4) into the bottom of 5 ml round bottom tube containing 1 mL of pre-equilibrated chemically defined medium (CDM), J. Anim. Sci. 2000. 78:152-157) containing about 2 mM caffeine and heparin (FCDM), and incubated in a 5% CO₂ incubator. The part of the frozen straw can be slanted at about 45 degree for about 20 min. After swim-up of the sperm cells, 0.5 ml of supernatant can be transferred into a 15-ml centrifugation tube containing 2 mL of FCDM and washed at 300 g for 5 min. The supernatant can be removed, and the pellet of sex-selected equine sperm cells (22) or the pellet of the equine sperm cells (4) can then be placed in an incubator until use.

Equine sex-selected ICSI can further include an equine oocyte(s) (56) obtained from a female equine mammal (57) (also referred to as a “donor mare”) by oocyte collection which includes utilizing one and half milligrams (“mg”) of GnRH analogue (for example Deslorelin; Betpharm, Lexington, Ky.) and 7.5 mg of recombinant equine luteinizing hormone (Aspen Biopharma Inc, Castle Rock, Colo.) administered to donor mares (57) when the following criteria were observed: 1) follicle >35 mm (average of length and width), 2) uterine edema, and 3) relaxed tone of the uterus and cervix. Deslorelin can then be administered and recombinant equine luteinizing hormone can be administered between about four and about five hours subsequent (for example if the Deslorelin is administered a 1 p.m. then the recombinant equine luteinizing hormone can be administered at between about 5 P.M. and 6 P.M.) to initiate follicular and oocyte maturation in vivo. Equine oocytes (56) can then be collected between 20 and 24 hours after administration of luteinizing hormone.

Specifically, transvaginal, ultrasound guided follicular aspirations using a linear ultrasound transducer (Aloka Co. Ltd., Wallingford, Conn.) and a 12-gauge double-lumen collection needle (Cook Veterinary Products, Spencer, Ind.) can be utilized. Before aspirations, donor mares (57) can be sedated (xylazine HCl; 0.4 mg/kg, i.v.; Vedco, Inc., St. Joseph, Mo. and butorphanol tartrate; 0.01 mg/kg, i.v.; Fort Dodge Animal Health, Fort Dodge, Iowa). Propantheline bromide (0.05 mg/kg, i.v.; Sigma Chemical Co., Saint Louis, Mo.) can be administered to relax rectal tone. The ultrasound transducer can be placed in a plastic casing that contains a needle guide (Aloka Co., Ltd.) and inserted into the anterior vagina. The ovary can then be positioned per rectum to image the preovulatory follicle. The aspiration needle can be advanced through the walls of the vagina and preovulatory follicle. Contents of the follicle are gently aspirated (150 mmHg) using a pump (Cook Veterinary Products) while the follicle is flushed with 100 mL of flush medium (EmCare complete embryo flush solution; ICP, Auckland, New Zealand) supplemented with 10 IU/mL of heparin (Calbiochem; La Jolla, Calif.) at 38.5° C.

Equine oocyte(s) (56) can then be immediately identified, washed, and placed in culture medium (TCM-199; Bio Whittaker; Walkersville, MD) with 10% fetal calf serum, 0.2 mM pyruvate, and 25 ug/mL gentamicin sulfate. Equine oocyte(s) (56) can then be incubated in an atmosphere of 6% CO₂ in air at 38.5° C. At the completion of culture equine oocytes (56) are stripped of cumulus cells in GMOPS (Vitrolife, Sweden) containing 200 IU/ml hyaluronidase (Sigma-Aldrich, MO, USA). Upon removal of cumulus, equine oocytes (56) were returned to culture medium until ICSI.

Equine ICSI can be performed between 38 and 40 hours after administration of Deslorelin to the equine oocyte donors. A piezo injection system (PMM Inc, Japan) can be used for injecting equine oocyte(s) (56) with a sex-selected equine sperm cell (22) isolated as above described. The outer diameter of a suitable sperm-injection pipette can be 5 μm. The holding pipette can have an outer diameter of about 120 to about 140 μm. Immediately before injection of the sex-selected equine sperm cell (22), 1 μL of sex-selected equine sperm cell (22) (or control equine sperm cell) suspension can be placed in a 5 μL GMOPS (Vitrolife, Sweden) containing 5% (w/v) polyvinylpyrrolidone (PVP)(ICN Biomedicals, OH, USA) under oil (Vitrolife, Sweden). Injection of sex-selected equine sperm cells (22) (or control equine sperm cells (4)) was carried out in a 40 μL drop of GMOPS containing an equine oocyte (56). Each sex-selected equine sperm cell (22) can be immobilized by applying a few pulses with the piezo drill and scoring the sperm tail. The sex-selected equine sperm cell (22) scored can be washed once in a clean 5% PVP drop before injection. All manipulations can be performed at about 30° C. room temperature.

Again referring to FIG. 1, the inventive sex-selected equine ICSI can further include a sex-selected equine embryo (58) (also referred to as “an equine embryo of a selected sex”) produced using the above-described steps of equine ICSI. The sex-selected equine embryo (58) can be cultured in 50 μl drops of pre-equilibrated DMEM/F12 medium (Sigma-Aldrich, MO, USA) with 10% fetal calf serum covered with mineral oil. Zygotes can be cultured individually at 38.5° C. under 5% CO₂, 5% O₂ and 90% N₂. Fertilization can be evaluated by evaluating cleavage under a microscope at 24 h and 48 h post-ICSI. Cleaved sex-selected equine embryos (58) can be cultured in the same condition for 7 days up to blastocyst stage, replacing culture medium every 3 days.

The inventive sex-selected equine ICSI can further include a recipient animal (59) capable of receiving a sex-selected equine embryo (58) cultured for a period of about 24 hours to about 48 hours post-ICSI. Typically, the recipient animal (59) will be a synchronized recipient mare to which a single sex-select equine embryo (58) can be surgically transferred into the oviduct. Oviducts of the recipient animal (59) can be exposed through standing flank laparotomies, and sex-selected equine embryos (58) can be transferred to the side contralateral ovulations of the recipient animal (59). Recipients can be placed in stocks for administration of a presurgical sedative (xylazine HCl, 0.3 mg/kg, and butorphanol tartrate, 0.01 mg/kg, i.v.). The surgical area can be clipped, scrubbed, and blocked with 2% lidocaine. Prior to surgery, recipient animals (59) can be given additional sedation (detomidine hydrochloride, 9 mg/kg, and butorphanol tartrate, 0.012 mg/kg, i.v.). An incision can be made through the skin approximately midway between the last rib and tuber coxae, and the muscle layers separated by blunt dissection. The ovary and oviduct can be exteriorized through the incision. The infundibullar os of the oviduct can be located, and embryo in ≦0.2 mL of GMOPS containing 0.5% BSA was transferred by advancing a fire-polished glass pipette approximately 2 to 3 cm into the oviductal lumen. Recipient animals (59) can receive phenylbutazone (2 g daily) at the time of surgery (i.v.) and for two additional days (p.o.). Antibiotics (Penicillin G procaine, 20,000 IU/kg, i.m. daily; Vedco, Inc.) can be administered before surgery and for 5 days after transfers. Regumate (2.2 mg/kg; Intervet Inc, KY, USA) can be supplemented one day after surgery every 24 hours until pregnancy examination, and was continued until 100 days for the pregnant mares.

For the transfer of blastocyst stage sex-selected equine embryos (58), a single sex-selected equine embryo (58) can be non-surgically transferred into the uterus of a synchronized recipient animal (59). GMOPS containing 0.5% BSA can be used as the transfer medium. Ultrasound examinations of uteri of sex-selected equine ICSI embryo recipients (5) for pregnancy were performed on days 12, 14, and 16 after transfer to determine presence of embryonic vesicles.

Now referring to Table 1 which shows the outcome of 42 equine oocytes intracytoplasmically injected with thawed unsex-selected equine sperm cells.

TABLE 1
ICSI with Unsex-selected Frozen Sperm Cells.

| No. oocytes | | No. embryo | |
|---|---|---|---|
| injected | No. cleaved | recipient | No. pregnant mare |
| 42 | 34 (81%) | 22 | 12 (54.5%) |

Now referring to Table 2 which shows the outcome of eight equine oocytes of the swim-up procedure and the outcome of twelve equine oocytes of the washing procedure intracytoplasmically injected with sex-selected equine sperm cells.

TABLE 2
ICSI With Sex-selected Frozen Sperm Cells.

| Sperm processing | No. oocytes | | No. pregnant/embryo |
|---|---|---|---|
| method | injected | No. cleaved | recipient |
| Swim-up | 8 | 5 (62.5%) | 1/1 (100%) |
| Washing | 12 | 3 (25%) | 0/1 (0%) |

The data set out in Table 1 and Table 2 evidences that sex-selected equine ICSI can be utilized to produce sex-selected equine embryos (58) which can be transferred to an recipient animal (59) to generate viable equine pregnancies from which live sex-selected equine foals (60) can be produced. The swim-up procedure can be used with greater success than washing alone to produce live foals from sex-selected frozen equine sperm cells obtained from prior frozen thawed equine sperm cells and the ICSI procedure set forth above.

Again referring to FIGS. 1 and 3, the inventive sex-selected equine ICSI can further include a live sex-selected equine foal (60). The live sex-selected equine foal (60) can have a sex predetermined by either injecting equine oocytes (56) with X chromosome bearing sperm cells (17) or with Y chromosome bearing sperm cells (18) which are the product of sorting or otherwise separating a plurality of equine sperm cells (4) into separate X-chromosome bearing and Y-chromosome bearing populations (6) (7). While FIG. 1 provides a block diagram which shows the general steps of the inventive method, it is not intended that the all embodiments of the invention be limited to the steps shown. Rather, the Figure provides a block diagram of the best mode or a preferred mode of the invention which can further include any of the additional steps, elements or equivalents of those steps or elements described herein.

While sex-selected equine sperm cells for the ICSI procedures described were obtained by flow cytometry, it is not intended that the invention be so limited and other methods of sex-selecting equine sperm cells can be used with the ICSI procedure described to produce sex-selected equine embryos. Additionally, while methods for the production of equine ICSI embryos are specifically described the methods can be utilized with sex-selected sperm cells (1) of other species of male mammals (3) to produce the corresponding sex-selected ICSI embryos (19) which can be transferred to recipient animals (59) capable of production of other species of sex-selected offspring (21).

As can be easily understood from the foregoing, the basic concepts of the present invention may be embodied in a variety of ways. The invention involves numerous and varied embodiments of an inventive equine ICSI and sex-selected equine ICSI and methods of using embodiments of the inventive equine ICSI and sex-selected equine ICSI to produce sex-selected equine ICSI embryos, sex-selected equine ICSI embryo recipients, and sex-selected equine ICSI offspring. While a particular source or sources of the various elements of the inventive sex-selected equine ICSI are identified through out this description; however, the invention is not so limited. Rather, these particular sources are provided as examples of the numerous and varied sources from which the elements of the invention can be obtained so that a person of ordinary skill can make and use the invention. Similarly, while particular methods are described including particular formulations and amounts, it is to be understood that these particular formulations and amounts provide an example of the best mode or a preferred mode of making and using the invention, the invention is not so limited and formulations and amounts which provide equivalent or similar results can be accomplished using methods similar to those described and are intended to be encompassed as embodiment of the invention.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “flow sorter” should be understood to encompass disclosure of the act of “flow sorting”—whether explicitly discussed or not—and, conversely, were there effectively disclosure of the act of “flow sorting”, such a disclosure should be understood to encompass disclosure of an “flow sorter” and even a “means for flow sorting.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the sex-selected equine ICSI products herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The Background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A method of producing a viable equine embryo of a selected sex, comprising the steps of:
a) obtaining a plurality of equine sperm cells of a male equine mammal;
b) determining a sex of said plurality of equine sperm cells of said male equine mammal;
c) sorting each of said plurality of equine sperm cells of said male equine mammal based upon determined said sex;
d) collecting each of said plurality of equine sperm cells of same said sex in a discrete container;
e) immobilizing one of said plurality of equine sperm cells of said selected sex;
f) obtaining an equine oocyte of a female equine mammal;
g) injecting the immobilized one of said plurality of equine sperm cells of said selected sex into said equine oocyte of said female mammal; and
h) producing said viable equine embryo of said selected sex.

2. The method of producing a viable equine embryo of a selected sex as described in claim 1, further comprising the step of obtaining a recipient animal capable of receiving said viable equine embryo of said selected sex.

3. The method of producing a viable equine embryo of a selected sex as described in claim 2, further comprising the step of transferring said viable equine embryo of said selected sex to said recipient animal.

4. The method of producing a viable equine embryo of a selected sex as described in claim 1, further comprising the step of freezing said plurality of sperm cells of same said sex collected in said discrete container.

5. The method of producing a viable equine embryo of a selected sex as described in claim 4, further comprising the step of providing said discrete container in the form of an artificial insemination straw.

6. The method of producing a viable equine embryo of a selected sex as described in claim 4, further comprising the step of thawing said plurality of sperm cells of same said sex frozen in said discrete container.

7. The method of producing a viable equine embryo of a selected sex as described in claim 6, further comprising the step of washing said plurality of sperm cells of same said sex prior to said step of immobilizing one of said plurality of equine sperm cells of said selected sex.

8. The method of producing a viable equine embryo of a selected sex as described in claim 7, wherein said step of washing said plurality of sperm cells of same said sex prior to said step of immobilizing one of said plurality of equine sperm cells of said selected sex comprises the step of washing said plurality of equine sperm cells of same said sex prior to said step of immobilizing one of said plurality of equine sperm cells of said selected sex in FCDM.

9. The method of producing a viable equine embryo of a selected sex as described in claim 7, further comprising the step of swimming up of said plurality of sperm cells of same said sex prior to said step of washing said plurality of sperm cells of same said sex.

10. The method of producing a viable equine embryo of a selected sex as described in claim 7, wherein said step of swimming up of said plurality of sperm cells of same said sex comprises the step of swimming up of said plurality of sperm cells of same sex in CDM containing about 2 mM caffeine and heparin.

11. The method of producing a viable equine embryo of a selected sex as described in claim 7, further comprising the step of suspending a portion of said plurality of sperm cells of same said sex in an amount of GMOPS containing about five percent PVP.

12. The method of producing a viable equine embryo of a selected sex as described in claim 11, further comprising the step of washing said immobilized one of said plurality of equine sperm cells of said selected sex in about five percent PVP.

13. The method of producing a viable equine embryo of a selected sex as described in claim 12, comprising the step of entraining said oocyte in a drop of GMOPS prior to said step of injecting an immobilized one of said plurality of equine sperm cells of said selected sex into said equine oocyte of said female mammal.

14. The method of producing a viable equine embryo of a selected sex as described in claim 13, further comprising the step of utilizing a piezo injection system for injecting an immobilized one of said plurality of equine sperm cells of said selected sex into said equine oocyte of said female mammal.

15. The method of producing a viable equine embryo of a selected sex as described in claim 14, further comprising the step of providing a sperm-injection pipette having an external diameter of about five micrometer.

16. The method of producing a viable equine embryo of a selected sex as described in claim 15, wherein said step of transferring said equine embryo of said selected sex to said recipient animal comprises the step of transferring said equine embryo of said selected sex to the oviductal lumen of said recipient animal.

17. The method of producing a viable equine embryo of a selected sex as described in claim 15, wherein said step of transferring said equine embryo of said selected sex to said recipient animal comprises the step of transferring said equine embryo of said selected sex to the uterus of said recipient animal.

18. The method of producing a viable equine embryo of a selected sex as described in claim 1, further comprising the step of providing a flow cytometer which performs said step of determining a sex of said plurality of equine sperm cells of said male equine mammal based on an amount of DNA in each of said plurality of equine sperm cells.

19. The method of producing a viable equine embryo of a selected sex as described in claim 18, further comprising the step of staining an amount of DNA in each of said a plurality of equine sperm cells of a male equine mammal with a DNA selective material for a period of time which provides substantially uniform staining of said amount of DNA.

20. The method of producing a viable equine embryo of a selected sex as described in claim 19, further comprising the step of electroporating said plurality of equine sperm cells of said male equine mammal in said DNA selective material.

21. The method of producing a viable equine embryo of a selected sex as described in claim 20, further comprising the steps of exposing said plurality of equine sperm cells of said male equine mammal containing said amount of DNA stained with said DNA selective material to a light source to generate an amount of fluorescence which varies based on the amount of said DNA stained with said DNA selective material.

22. The method of producing a viable equine embryo of a selected sex as described in claim 21, further comprising the step of detecting said amount of fluorescence which varies based on the amount of said DNA stained with said DNA selective material.

23. The method of producing a viable equine embryo of a selected sex as described in claim 21, further comprising the step of maturing said equine oocyte in vivo.

24. A foal having a selected sex produced by the method of claim 1.
